(19) United States
(12) Patent Application Publication
(10) Pub. No.: US 2009/0216154 A1
(43) Pub. Date: Aug. 27, 2009

(54) Automatically retractable safety blood sampler

(75) Inventor: Lee Lin Lee, Taipei (TW)

Correspondence Address: BACON & THOMAS, PLLC, 625 SLATERS LANE, FOURTH FLOOR, ALEXANDRIA, VA, 22314-1176, US

(73) Assignee: Bencha International Group. Inc

(21) Appl. No.: 12/071,428

(22) Filed: Feb. 21, 2008

(51) Int. Cl. A61B 5/153

(52) U.S. Cl. 600/576

(57) ABSTRACT

This invention of the automatically retractable safety blood sampler comprises a needle with a needle hub, a needle tube, a rubber washer, and a protective sheath, a cap, a barrel, a spring, and a blood collection tube. With blood collected, the blood collection tube is used for coercing the needle head to move forward, driving a sliding movement between slides on the needle hub and the snap hook on the barrel via the spring's force to automatically complete retraction of the needle head.

### BACKGROUND OF THE INVENTION

I. Field of the Invention

This invention of the automatically retractable safety blood sampler is related to a safe device that keeps the needle head stressed with a spring's automatically retractable motion inside the barrel after a blood collection completed to protect a user's safety by reducing injuries of needle stab.

II. Description of the Prior Art

To control or clean various microbes within a human body and sift as well as examine diseases & symptoms, needles, in general, are used as a tool for injection and blood collection at a human body.

Nowadays, frequent blood collections with needles everywhere have resulted in numerous cases and non-negligible sequelae owing to injuries of needle stab, for instance, 20 odd diseases like AIDS and B-type or C-type hepatitis from blood infection through needle heads that cause enormous social costs for dealing with the sequential derivative problems. As blood samplers, the known conventional lancets or vacuum lancets after being employed will expose infected needle heads that easily cause injuries of needle stab without functions of protecting the medical staff.

Therefore, in addition to convenience in usage, the inventor, while actively researching safety lancets, deliberates a user's safety as a major objective in designing this safety lancet with an automatically retractable structure that retracts the needle head back inside the barrel to avoid injuries of needle stab to the medical staff because of exposure of infected needle points after blood collection.

### SUMMARY OF THE INVENTION

This invention of the automatically retractable safety blood sampler comprises a needle head with a hub, a needle tube, a rubber washer, and a protective sheath, a cap, a barrel, a spring, and a blood collection tube. With press from the blood collection tube, the needle head is able to retract into the barrel by the spring to reach a safer design.

The major objective of designing this automatically retractable safety blood sampler is to offer an automatically retractable needle head with a needle hub forming plural U-type slides at the end for sliding motion of plural snap hooks inside the hub end. With a spring around the needle hub, the spring's motion may drive snap hooks, moving inside slides of the needle hub to complete an automatically retractable motion when the blood collection tube pushes the needle head forward.

With an illustration of diagrams, the detailed description and technical content related to this invention is displayed as follows:

### DETAILED DESCRIPTION OF THE PREFERRED EMBODIMENTS

Firstly, refer to FIGS. 1, 2, 3 and 4 for 3-dimensional pictures of components. This invention of the automatically retractable safety blood sampler has a lancet that will automatically retract into the barrel after blood collection. The device comprises a needle head 13, a cap 10, a barrel 11, a spring 12 and a blood collection tube 16 wherein the needle head 13 has a needle tube 133 and a needle hub 131, accommodating the needle tube 133 that penetrates one end of the hub. The needle tube 133 can be either a single needle tube with both sharp points or two single sharp-point needle tubes installed on both sides of the needle hub 131. In one end, the lancet forms a hollow bulge with an embedded part 1321 and a rib 1322 for penetration of the needle tube 133 and insertion of the protective sheath 15 and, near the embedded part 1321, forms a circular retainer 132 with a washer groove 1320 around its circumstance wherein the spring 12 is deployed around the surface of the extruded needle hub 131 connected to the side of the retainer and plural inward-concaved locating grooves 134 surround some adequate positions at the end of the needle hub 131 to form an approximate U-type slide as a route for retraction of the needle head after connecting plural upward-slope ramps 135, plural inward-concaved spiral-slope rotation slides 136, and plural inward-concaved penetration slides 137. A rubber washer 14 is used for fitting of the retainer 132 and the inner walls of the barrel 11. A circular spring 12 with compressive/extendable functions is used for coercing the needle head to retract. As a tubular container with one end open, the blood collection tube 16 contains a gasket 160 at the open end to form a vacuum status. With the snap hook 111 that has plural slightly elastic hooks facing the center of a circle at one end, the barrel 11 accommodates to slides on the needle hub 131 for sliding of the U-type route. Furthermore, due to a fingergrip 112 at the other end, the hollow inner barrel 110 is able to prepare space for containing the needle head 13 and the spring 12. A cap 10 is to offer a protection for the needle head 13 and fit the snap hook 111 of the barrel 11 wherein the snap hook 111 cannot rebound and there is no retraction of the needle head 13 in the event that the cap 10 does not separate from the snap hook 111 of the barrel 11.

Referring to FIGS. 5 & 6 as diagrammatic illustrations for combination of the blood collection tube and the needle tube's lower point of the automatically retractable safety blood sampler, the lower point 1332 at one end of the needle head 13 is covered with a protective sheath 15 and contained inside the embedded part 1321 due to the rib 1322. During blood collection, the upper point 1331 of the needle tube 133 is stabbed into a person's vein first and the blood collection tube 16 with a vacuum status formed by a covered gasket 160 is plunged to the inner barrel 110. When the blood collection tube 16 is pushed forward, the protective sheath 15 manufactured with material of soft silica gel is contracted to let the lower point 1332 of the needle tube 133 penetrate the protective sheath 15 and the gasket 160 for completion of blood collection because of blood flow by pressure difference between a vein and the blood collection tube 16.

Further, refer to FIGS. 7, 8, 9 & 10 as diagrammatic illustrations for inward contraction of the blood collection tube as well as the needle head of the automatically retractable safety blood sampler. With blood collected and the needle tube 133 removed from a human body, a user can push the blood collection tube 16, coercing the needle 13 forward to exit from the front end of the barrel 11. At this moment in time, owing to recession of the compressed spring 12 drawn back to the opposite direction, the snap hook 111 buckling the locating groove 134 slightly opens toward both sides via tilted ramps 135, and falls into the inward-concaved tilted rotation slide 136. In this regard, the scope of the slightly opened snap hook 111 depends on adjustment of the high/low retaining wall 113. Next, with the upper rim of the retainer 132 of the needle head 13 reaching the stop ring 17 of the barrel and a forward movement of the blood collection tube 16 failing, a user is able to slightly loose the blood collection tube 16 that the spring 12 starts extending to drive rotation of the needle hub 131, entering into the penetration slide 137 for complete disconnection from the snap hook 111. After the blood collection tube 16 is extracted, the spring 12 can totally extend, driving the needle head 13 to contract inward. When force from the extended spring is balanced off by resistance formed between the rubber washer 14 around the retainer 132 and walls of the inner barrel 110 inside the barrel 11 and the spring 12 is completely strained, the needle 13 can be totally contracted inward within the inner barrel 110 and leave a safety space of circa 2 mm.

### BRIEF DESCRIPTION OF THE DRAWINGS

FIG. 1 is a 3-dimensional exploded view of components of the automatically retractable safety blood sampler.

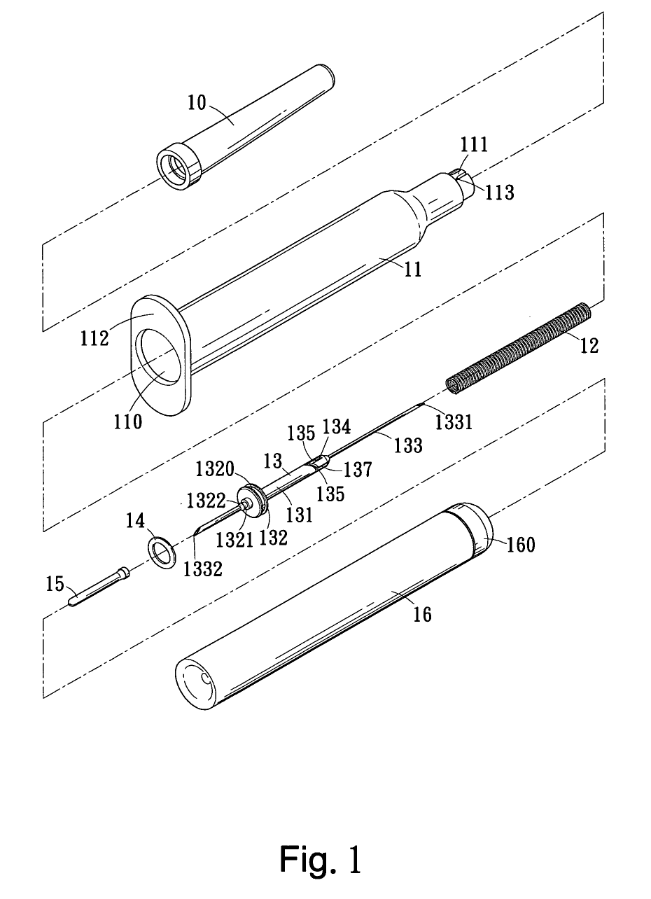

FIG. 2 is an exploded view for the needle head of the automatically retractable safety blood sampler.

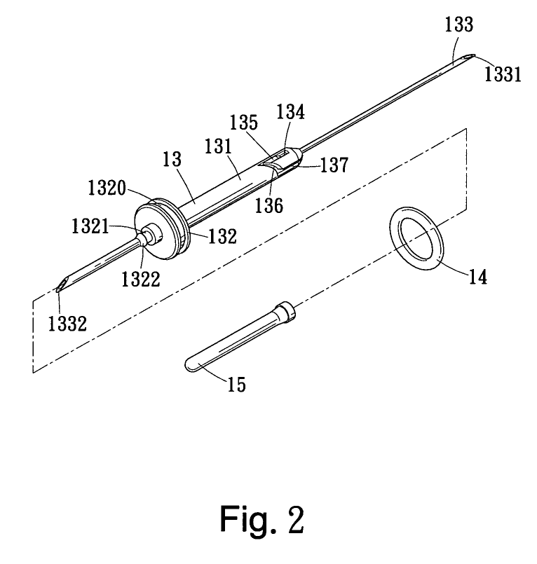

FIG. 3 is a cross-sectional view of the needle head embedded inside the barrel structure of the automatically retractable safety blood sampler.

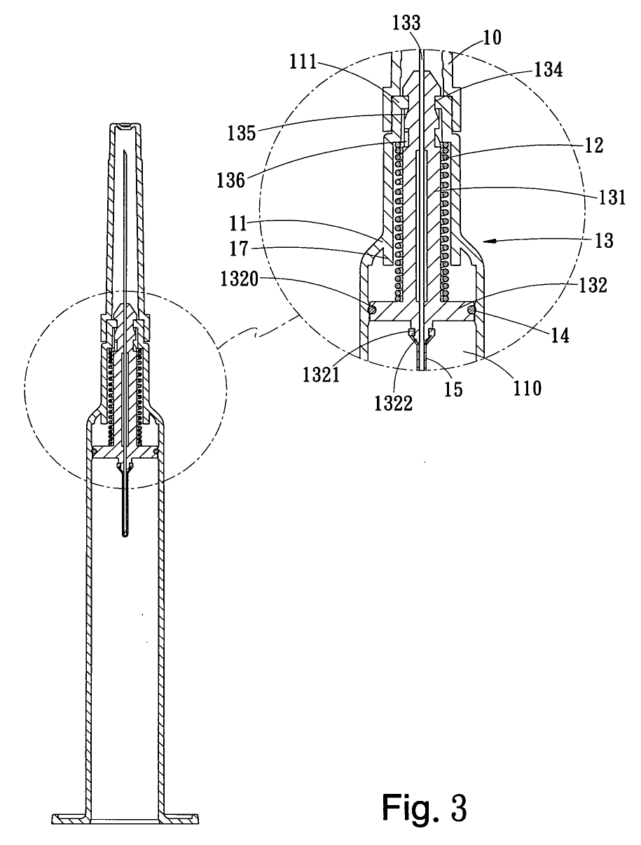

FIG. 4 is a cross-sectional view at the other end of the needle head of the automatically retractable safety blood sampler.

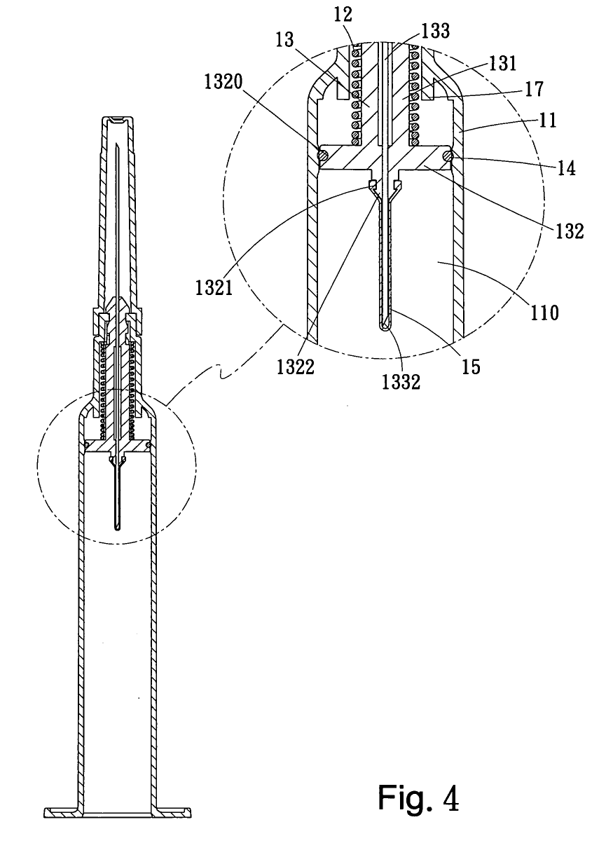

FIG. 5 is a plane view of combining components of the automatically retractable safety blood sampler.

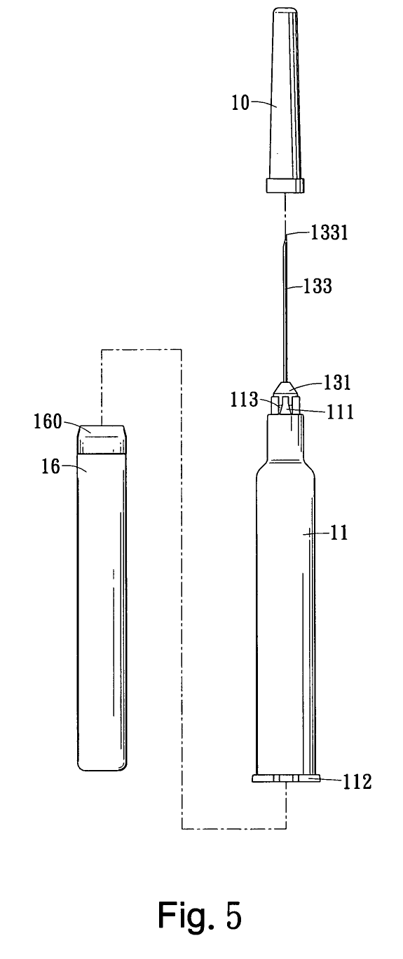

FIG. 6 is a diagrammatical illustration of combining the blood collection tube and the lower point of the needle head of the automatically retractable safety blood sampler.

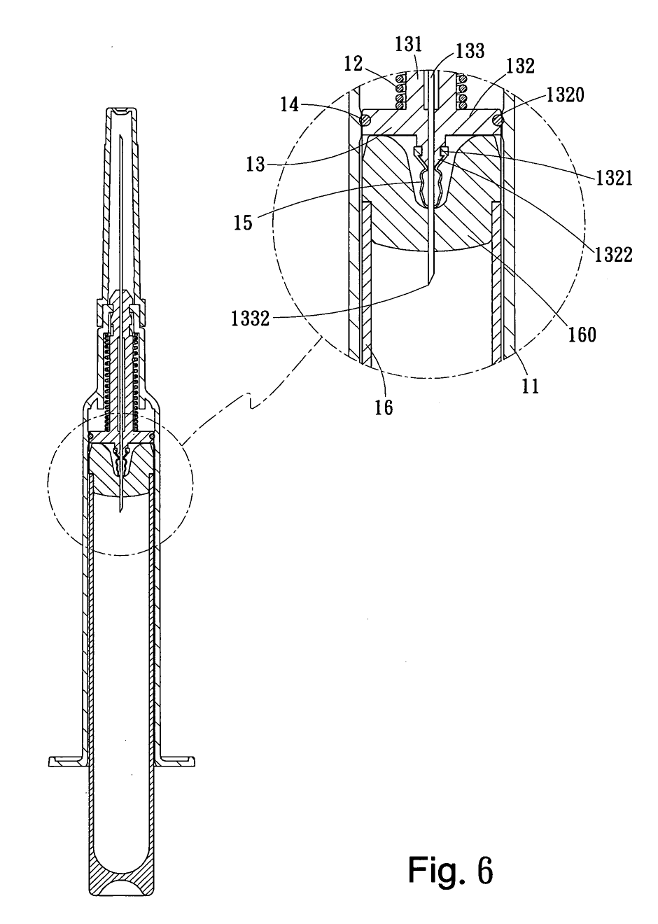

FIG. 7 is a diagrammatical illustration of fixing the needle hub and the snap hook of the automatically retractable safety blood sampler.

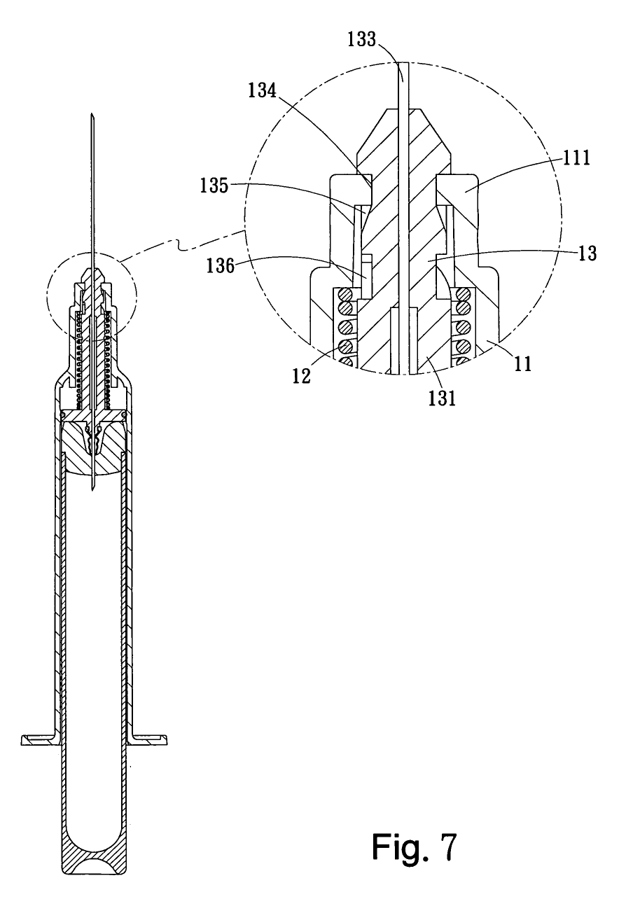

FIG. 8 is an operational view of embodiment for the automatically retractable safety blood sampler.

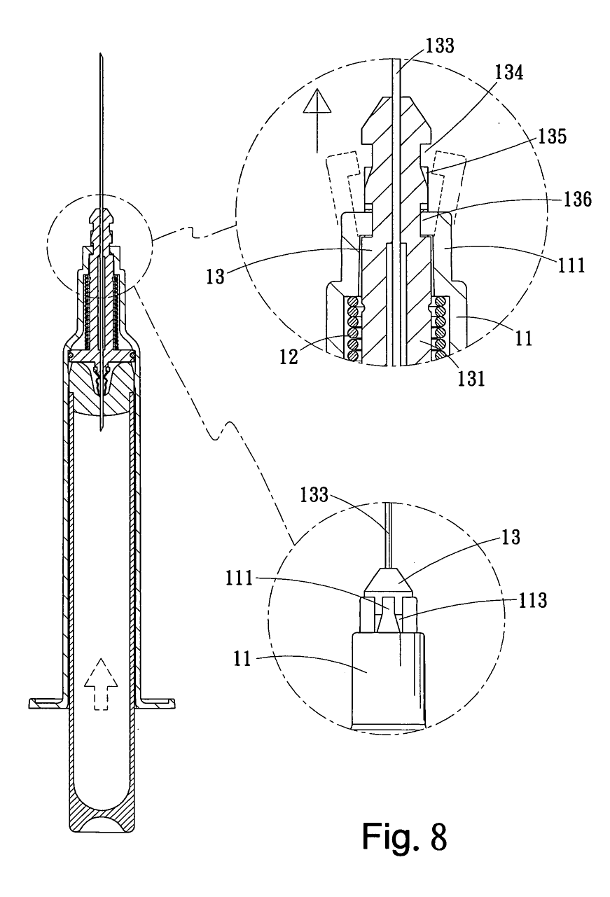

FIG. 9 is an operational view of another embodiment for the automatically retractable safety blood sampler.

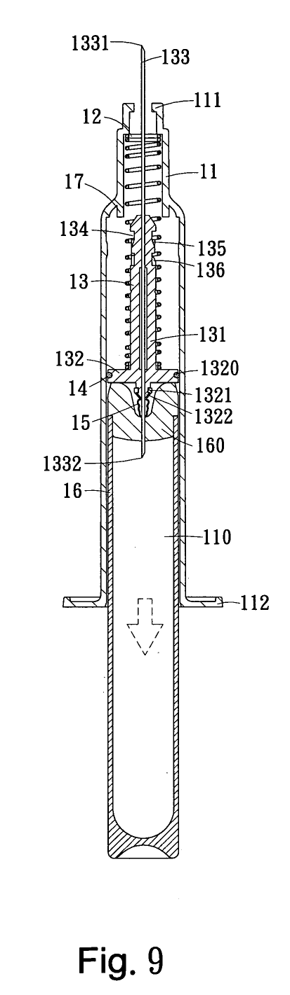

FIG. 10 is a diagrammatical illustration of the inward-contracted needle head for the automatically retractable safety blood sampler.

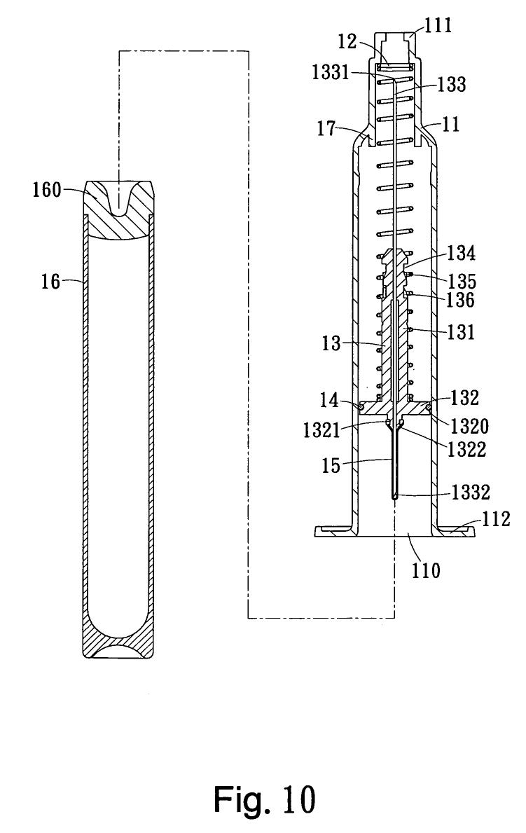

1.-3. (canceled)

4. An automatically retractable safety blood sampler, comprising:
a needle head having a needle hub with a needle tube disposed centrally therein, a protective sheath covering a first end of the needle tube, a rubber washer and a spring being fitted around the needle hub,
a barrel for receiving the needle head, wherein the barrel has a plurality of elastic snap hooks for securing the needle head, and a stop ring disposed on an interior portion of the barrel for controlling a distance of the needle head movement;
a cap removably engageable with the elastic snap hooks for capping the needle head;
a blood collection tube adapted for insertion into the barrel for collecting blood, a rubber gasket disposed at a first end portion of the blood collection tube for maintaining a vacuum in the blood collection tube,
wherein a first end portion of the needle hub has a plurality of inwardly concave locating grooves, a plurality of upwardly sloped ramps connecting respectively with the locating grooves, a plurality of inwardly concaved spirally sloped rotation slides connecting respectively with the sloped ramps, and a plurality of inwardly concaved penetration slides connecting respectively with the rotation slides such that the locating grooves, the sloped ramps, the rotation slides and the penetration slides form U-shaped slide routes which release the snap hooks for retracting the needle head within the barrel after blood collection.

5. The automatically retractable safety blood sampler of claim 4, wherein the needle tube can be embodied as either single or twin objects.
